(19) United States
(12) Patent Application Publication
(10) Pub. No.: US 2025/0290893 A1
(43) Pub. Date: Sep. 18, 2025

(54) GRAPHENE-BASED FET SENSOR ARRAY

(71) Applicant: IPLEXMED LDA,, Braga (PT)

(72) Inventors: Bruno Almeida, Porto (PT); Jérôme Borme, Porto (PT); Joana Rafaela Guerreiro, Porto (PT); Neide Vieira, Porto (PT)

(21) Appl. No.: 18/607,921

(22) Filed: Mar. 18, 2024

(51) Int. Cl. G01N 27/414; G01N 33/543

(52) U.S. Cl. CPC G01N 27/4145; G01N 33/5438

(57) ABSTRACT

This disclosure describes a system for fabricating and method for using a graphene-based FET (GFET) sensor array. The GFET sensor array is fabricated on a silicon substrate and comprises an array of sensors, a plurality of drain terminals, a source terminal, and a gate terminal. Each drain terminal of the plurality of drain terminals is configured to be electrically connected to a sensor of the array of sensors. The source terminal is configured to be electrically connected, via a common line, to each sensor of the array of sensors. The common line traverses the array of sensors in a serpentine pattern.

### BACKGROUND

Limitations and disadvantages of traditional sensors will become apparent to one of skill in the art, through comparison of such approaches with some aspects of the present method and system set forth in the remainder of this disclosure with reference to the drawings.

### BRIEF SUMMARY

Systems and methods are provided for producing a graphene-based FET sensor array, substantially as illustrated by and/or described in connection with at least one of the figures, as set forth more completely in the claims.

### BRIEF DESCRIPTION OF THE DRAWINGS

FIG. 1 illustrates a top view of an example GFET sensor array, in accordance with various example implementations of this disclosure.

FIG. 2 highlights the common source of the example GFET sensor array in FIG. 1, in accordance with various example implementations of this disclosure.

FIG. 3 highlights the gate of the example GFET sensor array in FIG. 1, in accordance with various example implementations of this disclosure.

FIG. 4 illustrates a close-up view of the example GFET sensor array in FIG. 1, in accordance with various example implementations of this disclosure.

FIG. 5A illustrates a top view of an example gate layer of a GFET sensor array, in accordance with various example implementations of this disclosure.

FIG. 5B illustrates a side view of a GFET sensor array with an example gate layer, in accordance with various example implementations of this disclosure.

FIG. 6A illustrates a top view of an example cap of a GFET sensor array, in accordance with various example implementations of this disclosure.

FIG. 6B illustrates a side view of a GFET sensor array with an example cap, in accordance with various example implementations of this disclosure.

### DETAILED DESCRIPTION

Biological sensors may be used to detect biomolecules such as nucleic acids, proteins, and cells that are associated with diseases. Sensors may include a transducer and a sample of bodily fluid that comprises a biological element such as an enzyme, an antibody or a nucleic acid. The biological element may interact with an analyte being tested and the biological response is converted into an electrical signal by the transducer.

A field-effect transistor (FET) may provide the transducer for converting biological responses into electrical signals. FETs have three terminals, namely a source, a drain and a gate. An electric field is applied at the gate terminal to measure the conductivity of a channel placed between source and drain.

Graphene and graphene derivatives possess superior characteristics (e.g., specific surface area, electronic properties, electron transport capability and flexibility) for various types of sensor channels. Graphene is biocompatible. The “on” and “off” stage of the transistor gate can be “on” if a biological signal is present, and “off” if not present. This may provide a direct connection between the complex but precise molecular signals involved in biology with a processor's logical calculation abilities. For example, graphene-based FET (GFET) sensors may be used for detecting nucleic acids, bacteria, virus, and/or fungal agents. A GFET sensor comprises a graphene channel between two electrodes with a gate contact to modulate the electronic response of the channel. The graphene may be exposed to a biological sample to enable binding of receptor molecules to the channel surface. Since the charge carrier mobility of graphene is very high, the change of conductance with respect to the presence of charged molecules of a GFET sensor is extremely abrupt which essentially aids the detection of extremely very amounts of the nucleic acids, bacteria, virus and/or fungal agents.

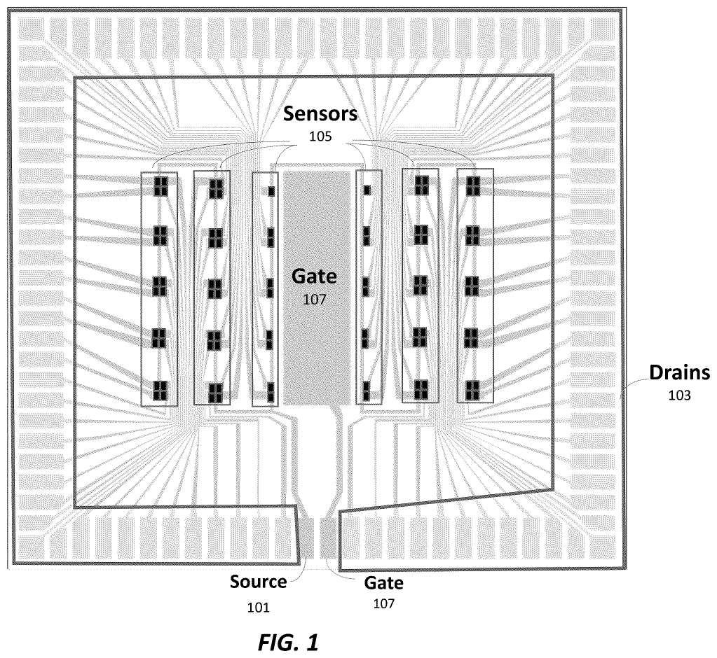

FIG. 1 illustrates a top view of an example GFET sensor array, in accordance with various example implementations of this disclosure. The terminals of the GFET sensor array comprise a common source terminal 101, a common gate terminal 107 and 98 drain terminals 103. Each of the 98 drain terminals 103 is connected to a graphene sensor 105. The sensors 105 are in 20 groups of 4 sensors, 8 groups of 2 sensors, and 2 individual sensors. Note that this particular grouping of exactly 98 sensors 105 is an example design. A smaller or larger number of sensors is also within the scope of this disclosure.

The response of each sensor 105 may be measured by the corresponding drain 103. A reader may read all sensors 105 simultaneously, or in groups. The source 101 may apply, for example, 1 mV-100 mV of voltage to each of the sensors 105. The gate 107 may apply, for example, −3.3 V to +3.3 V to each of the sensors 105. The gate voltage may be, for example, a sawtooth signal, a symmetrical triangular signal, a tangent signal, or a hyperbolic tangent signal. Arbitrarily-shaped signals, such as an asymmetrical signal, a coarse/fine signal, a pulsed signal, or a pulse-width modulated signal with an arbitrary duty cycle, may also be used. Measurement of the voltages at the drains 103 may proceed with any combination/size of parallel groups. Drain currents may also be measured when the sensors 105 are read sequentially.

The sensor chip may comprise one or multiple contact pads dedicated to the creation of a memory element. For example, one or more digits may be recorded to identify the chip. These one or more digits may also function as a checksum, authenticating recorded data.

A contact pad adjacent to the source 101 or the gate 107 may be connected to the source 101 or the gate 107 via a thin film resistor made of a line of the same metal used in the sensor current lines. The line may be of different lengths following a space-filling curve. The different line lengths give rise to different values of electrical resistance that define a multilevel resistive mask ROM. One multilevel cell may be defined per pad. This cell may be measured by the electrical resistance between the source 101 or the gate 107 and the adjacent pad defining the memory. Because of the fixed resistive nature of the mask resistor device, this measurement is not influenced by the presence or absence of the biological sample.

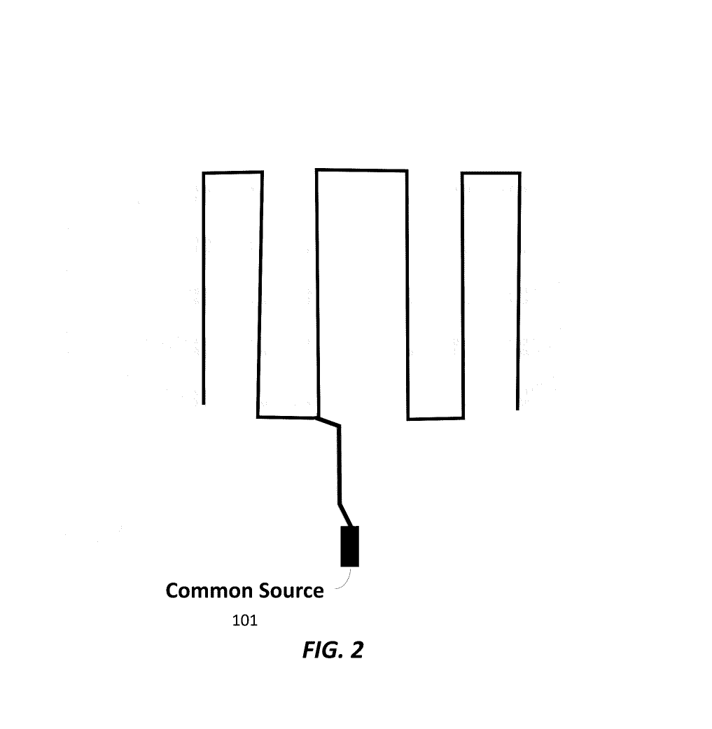

FIG. 2 highlights the common source 101 of the example FET sensor array in FIG. 1. The common source electrode 101 is designed as a serpentine to increase sensor density. The serpentine electrode 101 may be connected to one or multiple electrical pads along its length. The multiple connection enables to reduce the electrical resistance of the source connection. This design allows a small footprint (e.g., 5.5 mm) and a moderate contact pitch (e.g., 0.25 mm).

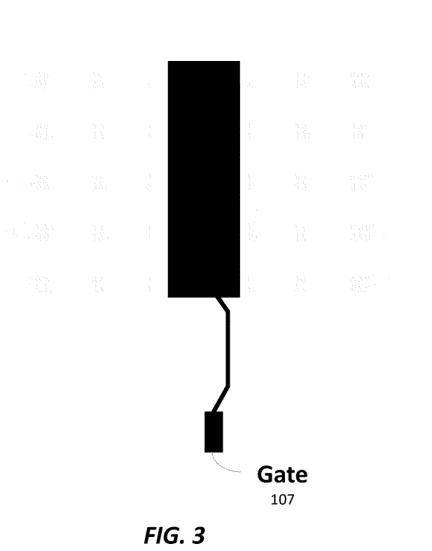

FIG. 3 highlights the central gate 107 of the example FET sensor array in FIG. 1.

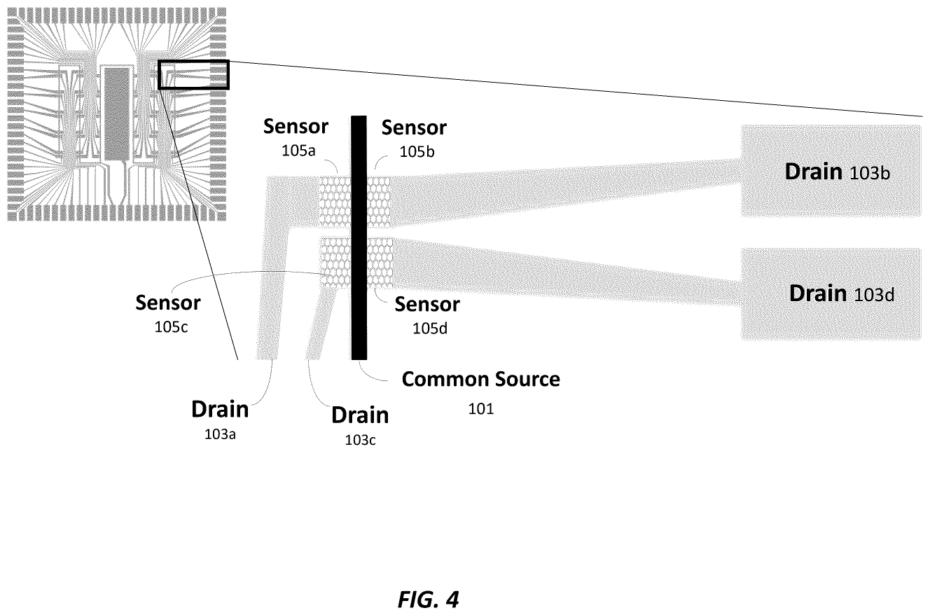

FIG. 4 illustrates a close-up view of the example FET sensor array in FIG. 1. FIG. 4 highlights a group of 4 sensors 105a, 105b, 105c and 105d. Each sensor 105a, 105b, 105c and 105d is operably coupled to the common source 101 and a respective drain 103a, 103b, 103c and 103d.

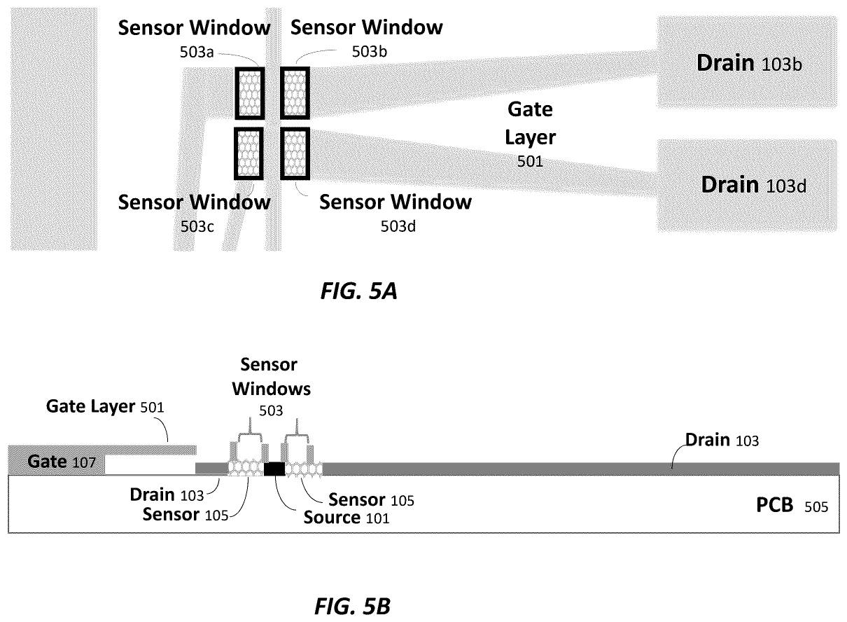

FIG. 5A illustrates a top view of an example gate layer of a FET sensor array, in accordance with various example implementations of this disclosure.

A gate layer 501 adds a continuous plane with windows 503a, 503b, 503c and 503d to 4 sensors, such as for example, sensors 105a, 105b, 105c and 105d of FIG. 4. The gate layer 501 may be made of gold or another metal with similar properties.

The gate electrode layer 501 may be added as a last fabrication step on top of an insulating layer (e.g., silicon oxide, silicon nitride, aluminum oxide, hexagonal boron nitride, or combinations thereof). The gate electrode layer 501 may cover the entire available area, except for the sensor windows 503a, 503b, 503c and 503d that expose the graphene channels. The sensor windows 503a, 503b, 503c and 503d allow the application of biological samples to the respective sensors 105a, 105b, 105c and 105d. The perimeter of pads 101, 103, 107 in FIG. 1 may be covered or left exposed.

FIG. 5B illustrates a side view of a FET sensor array with an example gate layer 501, in accordance with various example implementations of this disclosure.

The top gate layer 501 is placed above the passivation and makes contact with the central gate. The gate layer 501 is connected from below with lines to the sensors 105. Rather than being coplanar, the gate layer 501 is placed on a different plane from the sensors 105, drains 103 and source 101 separated by an insulator.

The design of FIGS. 5A and 5B maximizes the area of the gate 107 while using a minimum amount of silicon substrate area of the PCB 505. The design of FIGS. 5A and 5B may reduce an exposure of silicon oxide/nitride to the samples being tested. The design of FIGS. 5A and 5B may also reduce undesired electrical drifts in the sensor measurements.

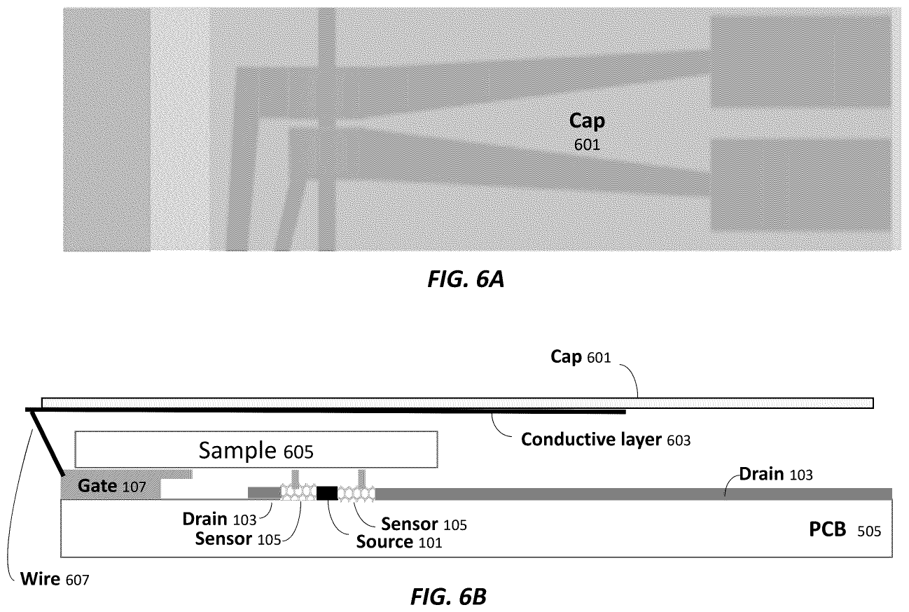

FIG. 6A illustrates a top view of an example cap 601 of a FET sensor array, in accordance with various example implementations of this disclosure. The cap 601 may be part of a separate chip that provides a cover/lid for the samples being tested.

FIG. 6B illustrates a side view of a FET sensor array with the example cap 601, in accordance with various example implementations of this disclosure.

The cap 601 may comprise a conductive thin film 603 that makes direct contact with the biological sample 605, through pressure or via a screw. The cap 601 mates with an enclosure of the cartridge/PCB 505 and contacts the gate 107 via a conductive wire 607 or other material.

The cap 601 may comprise, for example, glass, silicon, plastic, or aluminum. The conductive layer 603 may comprise a thin film of a noble metal such as gold or platinum or other a conductive material, such as conductive oxide or a transition metal nitride. The cap 601 may be coated with the conductive layer 603 using any manufacture techniques such as spray coating or physical vapor deposition. The cap structure 601, 603 may be suspended above the graphene sensors 105 until the sample 605 is added. The cap 601 may be cut or manufactured into a rectangle, a square or a round shape.

The cap 601 does not use the silicon area on the sensor chip. The array may comprise a chamber/well that enables a thin uniform distribution of the biological sample 605 onto the sensor area 105. The cap 601 may reduce or eliminate evaporation, which could cause a signal drift. The cap 601 may also provide a better distribution of an electric field.

While the present method and/or system has been described with reference to certain implementations, it will be understood by those skilled in the art that various changes may be made and equivalents may be substituted without departing from the scope of the present method and/or system. In addition, many modifications may be made to adapt a particular situation or material to the teachings of the present disclosure without departing from its scope. Therefore, it is intended that the present method and/or system not be limited to the particular implementations disclosed, but that the present method and/or system will include all implementations falling within the scope of the appended claims.

What is claimed is:

1. A system, the system comprising:
a silicon substrate covered in an insulating layer, configured with:
an array of sensors;
a plurality of drain terminals; and
a source terminal, wherein:
each drain terminal of the plurality of drain terminals is configured to be electrically connected to a sensor of the array of sensors,
the source terminal is configured to be electrically connected, via a common line, to each sensor of the array of sensors, and
the common line traverses the array of sensors in a serpentine pattern.

2. The system of claim 1, wherein the array of sensors comprises graphene sensors.

3. The system of claim 1, wherein a sensor of the array of sensors is configured to form a channel between the source terminal and one drain terminal of the plurality of drain terminals.

4. The system of claim 1, wherein a conductivity of a sensor of the array of sensors is determined when an electric field applied at a gate terminal.

5. The system of claim 1, wherein a conductivity of a sensor of the array of sensors is determined according to a biological sample applied to the sensor.

6. The system of claim 1, wherein the system comprises a gate layer on a plane above the silicon substrate.

7. The system of claim 6, wherein the gate layer and a sensor, of the array of sensors, are configured to be electrically connected via a biological sample applied to the sensor.

8. The system of claim 6, wherein the gate layer is configured to be electrically connected to a terminal on the silicon substrate.

9. The system of claim 6, wherein the gate layer comprises a plurality of windows that allow a biological sample to be added to a sensor of the array of sensors.

10. The system of claim 6, wherein:
system comprise a cap that covers the array of sensors, and
the gate layer is applied to the cap.

11. A method, the method comprising:
adding a biological sample onto each sensor in an array of sensors on a silicon substrate;
applying a first electrical signal to a common source terminal on the silicon substrate, wherein the common source terminal is electrically connected, via a common line in a serpentine pattern, to each sensor of the array of sensors;
applying a second electrical signal to a common gate terminal on the silicon substrate, and
determining a conductivity of a sensor in the array of sensors at a corresponding drain terminal of a plurality of drain terminals.

12. The method of claim 11, wherein the array of sensors comprises graphene sensors.

13. The method of claim 11, wherein a sensor of the array of sensors is configured to form a channel between the common source terminal and one drain terminal of the plurality of drain terminals.

14. The method of claim 11, wherein the conductivities of two or more sensors in the array of sensors are determined in parallel.

15. The method of claim 11, wherein the conductivity of the sensor of the array of sensors is determined according to a current measured at the corresponding drain terminal.

16. The method of claim 11, wherein the gate terminal is electrically connected to a gate layer on a plane above the silicon substrate.

17. The method of claim 16, wherein the gate layer and a sensor, of the array of sensors, are configured to be electrically connected via the biological sample.

18. The method of claim 16, wherein the method comprise covering the silicon substrate with the gate layer.

19. The method of claim 16, wherein the biological sample is added onto each sensor of the array of sensors via a plurality of windows in the gate layer.

20. The method of claim 16, wherein the method comprises covering the silicon substrate with a cap that covers the array of sensors.
